(19) United States
(12) Patent Application Publication
(10) Pub. No.: US 2024/0091294 A1
(43) Pub. Date: Mar. 21, 2024

(54) Extract of Top Growth of Holy Basil, and Cosmetic or Dermatological Compositions Containing Same

(71) Applicant: Laboratoires Expanscience, Paris La Defense Cedex (FR)

(72) Inventors: Sophie LECLERE-BIENFAIT, Dreux (FR); Stephanie BREDIF, Croisilles (FR)

(73) Assignee: Laboratoires Expanscience, Paris La Defense Cedex (FR)

(21) Appl. No.: 18/272,461

(22) PCT Filed: Jan. 20, 2022
(86) PCT No.: PCT/FR2022/050111

§ 371 Date: Jul. 14, 2023

(30) Foreign Application Priority Data
Jan. 20, 2021 (FR) 2100513

(51) Int. Cl. A61K 36/53; A61K 8/9789; A61P 17/02; A61P 17/18; A61P 29/00; A61Q 17/04; A61Q 19/00; A61Q 19/08

(52) U.S. Cl. CPC A61K 36/53; A61K 8/9789; A61P 17/02; A61P 17/18; A61P 29/00; A61Q 17/04; A61Q 19/007; A61Q 19/08; A61K 2236/15; A61K 2236/333; A61K 2236/53; A61K 2800/522

(57) ABSTRACT

The invention relates to an extract of top growth of holy basil, in particular of Ocimum sanctum, a method for preparing the extract, and the extract obtainable by said method. The invention also relates to a composition comprising such an extract, the composition advantageously being a cosmetic, pharmaceutical or dermatological composition. The invention also relates to such a composition or such an extract for use in preventing or treating disorders or diseases of the skin, the mucous membranes or the skin appendages, and for use in preventing or treating vascular disorders. Finallyc the invention relates to a cosmetic care method for the skin, skin appendages or mucous membranes, with a view to improving the condition or appearance thereof, which method consists in administering such a composition or such an extract.

The invention relates to a composition comprising an extract of aerial parts of green or purple holy basil (Ocimum sanctum). The composition is advantageously cosmetic, pharmaceutical or dermatological. The invention also relates to a method of extracting an extract of the aerial parts of holy basil, and to the extract obtainable by said method. The invention also relates to such a composition or extract for use in the prevention or treatment of skin, mucous membrane or skin appendage disorders or pathologies, and in the prevention or treatment of vascular disorders. Lastly, the invention relates to a cosmetic care method for the skin, skin appendages or mucous membranes, in view of improving their condition or appearance, consisting in administering such a composition or such an extract.

### Holy Basil

### Botanical Description

Ocimum sanctum (or ocimum tenuiflorum), commonly known as “holy basil” or “tulsi”, is an aromatic plant in the Lamiaceae family. It grows 30 to 60 cm tall, with hairy stems. Leaves are green or purple, depending on the variety. They are simple, petioled and up to 5 cm long. The small, purplish flowers are placed on elongated racemes. There are two varieties of holy basil: green basil and purple basil. Both varieties have a similar phytochemical composition.

Holy basil is cultivated in Asia for its religious and medicinal uses, particularly in Ayurvedic medicine, where it is considered an “elixir of life”. Holy basil leaves are also used in Thai cooking and as an insect repellent.

### Phytochemistry

Composition of Ocimum sanctum:

Proteins: 174.5 g/kg dry plant

Fibers: 90.7 g/kg dry plant

Ashes: 135.6 g/kg dry plant

Many molecules have been identified in aqueous and alcoholic extracts of Ocimum sanctum. Polyphenols are widely present, with rosmarinic acid as the main compound. Ursolic acid, apigenin derivatives and phenolic acids are also widely reported.

Sugars are composed of xylose and polysaccharides. Stems and leaves contain saponins, flavonoids, triterpenoids and tannins.

The mineral composition shows that potassium is in the majority (see Table 1):

TABLE 1
Mineral composition of Ocimum sanctum (ppm)

| Ca | P | Mg | K | Na | Cu | Zn | Mn |
|---|---|---|---|---|---|---|---|
| 1.00 | 10.90 | 1.05 | 5260 | 680 | 12.31 | 81.66 | 51.35 |

### DESCRIPTION OF THE INVENTION

The subject of the invention is a polyphenol-rich extract of holy basil, in particular a polyphenol-rich extract of the aerial parts of holy basil.

In the context of this invention, the terms “holy basil” and “Ocimum sanctum” have the same meaning and can be used interchangeably. There are two varieties of holy basil: green and purple. Unless otherwise specified, for the purposes of the present invention, “holy basil” or “Ocimum sanctum” refers to the two existing varieties, i.e., the green and purple varieties.

In the context of the invention, “aerial parts” of Ocimum sanctum include leaves, stems and flowers, preferably leaves and stems.

By “polyphenol-rich extract” we refer to an extract comprising predominantly or essentially polyphenols.

Thus, the extract according to the invention comprises at least 6% by weight of polyphenols, based on the total weight of the dry extract, i.e., at least 1.2 mg of polyphenols per mL of liquid extract (in gallic acid equivalent). Advantageously, the extract according to the invention comprises at least 9% by weight, more advantageously at least 11% by weight, more advantageously at least 15% by weight, of polyphenols, relative to the total weight of the dry extract. Advantageously, the extract according to the invention comprises between 6% and 17%, more advantageously between 9% and 17%, more advantageously between 11% and 17%, more advantageously between 15% and 17% by weight of polyphenols. The percentages are expressed relative to the total weight of said dry extract (before any addition of a drying medium) and are determined according to the Folin Ciocalteu method.

For the purposes of this invention, a polyphenol is a molecule comprising at least two hydroxyl functions carried by one or more benzene rings. Particularly advantageously, polyphenols according to the invention contain or consist of hydroxycinnamic derivatives and flavonoids.

Advantageously, the extract according to the invention comprises at least 5% by weight of hydroxycinnamic derivatives, more advantageously at least 7% by weight of hydroxycinnamic derivatives, i.e., at least 1.0 mg of hydroxycinnamic derivatives per mL of liquid extract. More advantageously, the extract according to the invention comprises at least 10% by weight, more advantageously at least 15% by weight, of hydroxycinnamic derivatives. Advantageously, the extract according to the invention comprises between 5% and 20% by weight, more advantageously between 7% and 20% by weight, more advantageously between 10% and 20% by weight, more advantageously between 15% and 20% by weight, of hydroxycinnamic derivatives. The percentages are expressed relative to the total weight of said dry extract (before possible addition of a drying medium), in chlorogenic acid equivalent and are determined according to the Arnow method.

Advantageously, the extract according to the invention comprises at least 2.5% by weight of flavonoids, expressed as rutin equivalent, relative to the total weight of said dry extract, more advantageously at least 3.5% by weight. Advantageously, the extract according to the invention comprises between 2.5% and 6% by weight, more advantageously between 3.5% and 6% by weight of flavonoids, expressed as rutin equivalent, relative to the total weight of said dry extract. The flavonoid content is determined using the Loots (AlCl₃) method.

Particularly advantageously, the extract according to the present invention comprises at least 6% by weight of polyphenols based on the total weight of the dry extract, i.e., at least 1.2 mg of polyphenols per ml of liquid extract (in gallic acid equivalent), in particular at least 5% by weight of hydroxycinnamic derivatives based on the total weight of the dry extract, and at least 2.5% by weight of flavonoids, expressed as rutin equivalent, relative to the total weight of the dry extract.

In the context of the present invention, hydroxycinnamic derivatives are molecules which include the following motif in their structure:

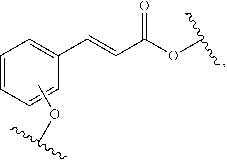

it being understood that the hydrogens of the motif can be substituted by any other group. Hydroxycinnamic derivatives within the meaning of the invention include notably hydroxycinnamic acids, esters and glycosylated derivatives.

Examples of hydroxycinnamic derivatives according to the present invention include chicoric acid, caffeic acid, rosmarinic acid or caffaric acid, and mixtures thereof.

In the context of the present invention, flavonoids are defined as molecules which include the following motif in their structure:

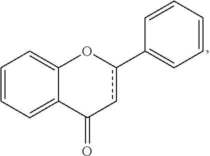

it being understood that the hydrogens of the moiety can be substituted by any other group. The dotted line in the above formula indicates a single or double bond.

Examples of flavonoids according to the present invention include apigenin, apigenin-7-glucuronide or luteolin-7-glucuronide, and mixtures thereof.

For the purposes of the present invention, flavonoids and hydroxycinnamic derivatives are typically polyphenols.

Particularly advantageously, the extract according to the invention is essentially devoid of eugenol and methyl eugenol, which are allergenic compounds and are phenols, not polyphenols in the meaning of the present invention. Eugenol and methyl eugenol are not classified in the polyphenol family, notably because of their physicochemical properties. In fact, these compounds are among the main constituents of holy basil essential oil and are steam-entrainable compounds, unlike the polyphenols of the present invention.

Thus, advantageously, the extract according to the present invention does not comprise eugenol and methyl eugenol or comprises less than 50 ppm eugenol and less than 100 ppm methyl eugenol.

Advantageously, according to the present invention, the extract is not an essential oil, as the constituents of the extract are not steam-entrainable.

In the context of the present invention, the polyphenol-rich extract described above is advantageously obtained by solid/liquid extraction of the aerial parts of holy basil, in a solvent chosen from water, glycols or glycerol, and mixtures thereof. The extraction solvent according to the invention is a non-toxic solvent and is more particularly chosen from binary mixtures water/glycols or water/glycerol, advantageously in a proportion of between 30% and 90%, typically between 50% and 80%, more advantageously between 60% and 70% glycol or glycerol in water, by volume relative to the total volume of solvent used (water+glycol or water+glycerol). In particular, the extraction solvent is chosen from the binary mixtures water/propanediol, water/propylene glycol, water/glycerine, especially water/propanediol, more particularly water/1,3-propanediol or water/glycerine.

Advantageously, the above-mentioned glycols are glycols of vegetable origin, in particular propanediol and/or propylene glycol, more particularly propanediol, especially 1,3-propanediol.

Advantageously, the above-mentioned glycerol is of vegetable origin.

Another object of the invention is a method for preparing a polyphenol-rich extract of holy basil aerial parts, comprising at least one solid/liquid extraction step in a suitable solvent mixture, in particular as defined in the above paragraph, and under optimum conditions of pH, time and temperature, known to the person skilled in the art.

Advantageously according to the invention, the method for preparing a polyphenol-rich extract of holy basil aerial parts comprises the following successive steps:
a) Grinding the aerial parts of holy basil;
b) Extracting ground aerial parts in a solvent selected from water, glycols, glycerol and mixtures thereof, advantageously selected from binary mixtures water/glycols or water/glycerol, under optimum conditions of time, pH and temperature;
c) Separating the solid phase from the liquid phase by decantation and/or centrifugation and/or successive filtration; and
d) Optionally, drying the extract obtained in step c).

Particularly advantageously according to the present invention, the extract preparation method contains a step for removing eugenol or methyl eugenol or substantially reducing the eugenol and methyl eugenol content, typically by vacuum evaporation or deodorization.

Step a) of grinding the plant can be carried out by methods known to the person skilled in the art, in particular using a cutter mill, a hammer mill, etc.

In step b), extraction is preferably carried out in the presence of an extraction solvent chosen from binary mixtures water/glycols or water/glycerol. Advantageously, the extraction solvent used in step b) is chosen from the binary mixtures water/glycols or water/glycerol in a proportion of between 30% and 90%, typically between 50% and 80%, more advantageously between 60% and 70% glycol or glycerol in water, by volume relative to the total volume of solvent used 30 (water+glycol or water+glycerol). In particular, the extraction solvent is chosen from the binary mixtures water/propanediol, water/propylene glycol or water/glycerine, especially water/propanediol, more particularly water/1,3-propanediol or water/glycerine.

Advantageously, the glycols are glycols of vegetable origin, in particular propanediol and/or propylene glycol, more particularly propanediol, especially 1,3-propanediol. Advantageously, glycerol is of vegetable origin.

In addition, step b) of solid/liquid extraction is preferably carried out at a temperature comprised between 20° C. and 90° C., in particular between 30° C. and 80° C., more particularly between 30° C. and 75° C., typically 60° C..

In contrast to other prior art methods, the extraction duration has the advantage of being short. It is advantageously between 30 minutes and 4 hours, in particular between 1 hour and 3 hours, advantageously it is around 2 hours.

Step c) for separating the solid phase from the liquid phase is carried out by methods known to the skilled person, in particular by decantation, centrifugation and/or successive filtrations, preferably by filtration. During step c), the liquid phase obtained is advantageously purified and concentrated, for example by ultrafiltration and/or sterilizing filtration. Step c) is carried out until the liquid is perfectly clear and microbiologically clean to a degree of less than or equal to 100 CFU/g total germ.

Advantageously, the polyphenol-rich extract according to the invention can be stabilized by drying step d), by methods known to the skilled person.

Step d) of drying can, for example, be carried out in the presence of a carrier such as maltodextrins or acacia fibers (Fibregum® by CNI). The carrier content typically varies according to a ratio ranging from 0% to 80% of carrier relative to the percentage of dry matter obtained in the liquid form of the extract.

The extract may be dried by freeze-drying to obtain a final powder. The final powder advantageously comprises 30 to 70% by weight of dry matter from the extract, the balance to 100% being the freeze-drying carrier. More advantageously, the final powder comprises 50% extract dry matter and 50% freeze-drying carrier.

Advantageously, the method according to the invention does not include step d). Thus, the extract as obtained is preserved in the extraction solvent, the solvent being in an amount sufficient to exhibit bacteriostatic/bactericidal activity, in particular the solvent being in an amount greater than or equal to 50% by weight relative to the total weight of extract and solvent.

The extraction method according to the invention advantageously makes it possible to obtain an extract concentrated in polyphenols in a single main extraction step, with a simple method to implement. In other words, it makes it possible to obtain concentrated extracts without multiplying successive extraction steps as in fractionation.

Preferably, by way of example, the polyphenol-rich extract according to the invention can be obtained according to the following method:

i. Grinding the aerial parts of holy basil;
ii. Dissolving ground aerial parts at 10% (w/w) in a 1,3-propanediol/water mixture (60/40) or in a glycerine/water mixture (60/40);
iii. Extracting under stirring for 2 hours at 60° C.;
iv. Purifying through successive filtration steps;
v. Advantageously removing eugenol and methyl eugenol, typically by vacuum evaporation or deodorization; and
vi. Sterile filtering.

In the description hereinafter, the expression “extract according to the invention” will be used to designate the extract as such, as defined above, or the extract obtainable by the method according to the invention as described above. The extract obtainable by the method described above has the same composition as the extract as such, as defined above.

Another object of the invention is a composition comprising an extract according to the invention and a water/solvent mixture in a water/solvent ratio (v/v) of between 50/50 and 0/100, advantageously between 30/70 and 10/90, more advantageously 20/80, said solvent being chosen from glycols, vegetable glycerine and mixtures thereof, preferably from glycols of vegetable origin, and preferably from 1,3-propanediol.

Advantageously, the composition comprises from 0.001 to 30% by weight, advantageously 0.001% to 10% by weight, of an extract according to the invention (expressed as weight of dry extract relative to the total weight of the composition) and between 70% and 99.999% by weight, advantageously between 90% and 99.999% by weight, of a water/solvent mixture, relative to the total weight of the composition, the water/solvent ratio being between 50/50 and 0/100, advantageously between 30/70 and 10/90, more advantageously 20/80, and the solvent being chosen from glycols, vegetable glycerine and mixtures thereof, preferably from glycols of vegetable origin, and preferentially from 1,3-propanediol. According to this aspect of the invention, the solvent is in an effective quantity for a physical and microbiological stabilizing action of the composition according to the invention and in particular of the extract according to the invention.

Another object of the invention is a composition comprising a polyphenol-rich extract of the aerial parts of Ocimum sanctum according to the invention, as active ingredient, and optionally a suitable excipient. The extract according to the invention is as defined in the paragraphs above concerning the extract as such and those concerning the extract obtainable by the method according to the invention.

The composition is advantageously cosmetic, pharmaceutical or dermatological. Said composition is preferably formulated for external topical administration.

Advantageously, the composition according to the invention comprises from 0.001 to 10%, typically from 0.01 to 5%, by weight of extract according to the invention, the weight of the extract being expressed as dry extract, relative to the total weight of the composition.

The composition according to the invention may also include one or more other active ingredients.

The composition according to the invention can be formulated in the form of various preparations suitable for topical administration and include in particular creams, emulsions, milks, ointments, lotions, oils, aqueous or hydroalcoholic or glycolic solutions, powders, patches, sprays, shampoos, varnishes or any other product for external application.

Depending on its nature (cosmetic, pharmaceutical or dermatological), the composition according to the invention may also comprise at least one cosmetically, pharmaceutically or dermatologically acceptable excipient. In particular, the composition according to the present invention may also comprise at least one cosmetically, pharmaceutically or dermatologically acceptable adjuvant known to the skilled person, chosen from surfactants, thickeners, preservatives, fragrances, colorants, chemical or mineral filters, moisturizing agents, thermal waters, etc. The skilled person knows how to adapt the formulation of the composition according to the invention using his or her general knowledge.

The optimal dosage and galenic form of the compositions according to the invention can be determined according to the criteria generally taken into account in establishing a pharmacological, dermatological or cosmetic treatment adapted to a patient or animal, such as the age or body weight of the patient or animal, the severity of its general condition, tolerance to treatment, side effects observed, skin type.

Another object of the invention is an extract according to the invention or a composition according to the invention for use in preventing and/or treating disorders or pathologies of the skin and/or mucous membranes (gums, periodontium, genital mucous membranes) and/or skin appendages (hair and nails), whether immature, normal or mature/aged, advantageously inflammatory reactions, oxidation reactions, disorders related to intrinsic or extrinsic stress (such as psychological stress, stress related to a state of anxiety, stress related to radical attacks related or not to pollution (particularly chemical or atmospheric pollution) and/or related to UV or IR exposure), barrier or homeostasis disorders, aging, particularly chronological and/or actinic aging, photosensitized skin, and/or mechanical or thermal aggressions.

Another object of the invention is an extract according to the invention or a composition according to the invention for use in preventing and/or treating vascular disorders, in particular redness and couperose.

Another object of the invention is an extract according to the invention or a composition according to the invention for use in preventing and/or treating imbalances and/or disorders related to the imbalance of the microbiota of the skin and/or mucous membranes (gums, periodontium, genital mucous membranes) and/or skin appendages, immature, normal or mature/aged. In fact, the extract according to the invention has a protective activity for the skin and/or mucous membranes (gums, periodontium, genital mucous membranes) and/or skin appendages and/or their adnexa, whether immature, normal or mature/aged, against mechanical, microbial, thermal and free-radical aggressions. The extract according to the invention acts to defend the microbiota and thus helps combat microbiota imbalance. The extract also helps stimulate the skin's immune defenses and antioxidant system.

Another object of the invention is the use of an extract according to the invention or a composition according to the invention for the manufacture of a cosmetic, pharmaceutical or dermatological composition for preventing and/or treating disorders or pathologies of the skin and/or mucous membranes (gums, periodontitis, genital mucous membranes) and/or skin appendages (hair and nails) whether immature, normal or mature/aged, advantageously inflammatory reactions, oxidation reactions, disorders related to intrinsic or extrinsic stresses (such as psychological stress, stress related to a state of anxiety, stress related to radical attacks related or not to pollution (particularly chemical or atmospheric pollution) and/or related to UV or IR exposure), barrier or homeostasis disorders, aging, particularly chronological and/or actinic aging, photosensitized skin, and/or mechanical or thermal aggressions.

Another object of the invention is the use of an extract according to the invention or of a composition according to the invention for the manufacture of a pharmaceutical, cosmetic or dermatological composition for preventing and/or treating vascular disorders, in particular redness and couperose.

Another object of the invention is the use of an extract according to the invention or of a composition according to the invention for the manufacture of a pharmaceutical, cosmetic or dermatological composition for preventing and/or treating imbalances and/or disorders related to the imbalance of the microbiota of the skin and/or mucous membranes (gums, periodontium, genital mucous membranes) and/or skin appendages and/or their adnexa, whether immature, normal or mature/aged.

A further object of the invention is a method for preventing and/or treating disorders or pathologies of the skin and/or mucous membranes (gums, periodontitis, genital mucous membranes) and/or skin appendages (hair and nails), whether immature, normal or mature/aged, advantageously inflammatory reactions, oxidation reactions, disorders related to intrinsic or extrinsic stresses (such as psychological stress, stress related to a state of anxiety, stress related to radical attacks related or not to pollution (particularly chemical or atmospheric pollution) and/or related to UV or IR exposure), barrier or homeostasis disorders, aging, in particular chronological and/or actinic aging, photosensitized skin, and/or mechanical or thermal aggressions, comprising the administration, in particular topical administration, of an effective amount of an extract according to the invention or a composition according to the invention, to a subject in need thereof.

Another object of the invention is a method for preventing and/or treating vascular disorders, in particular redness and couperose, comprising the administration, in particular topical administration, of an effective amount of an extract according to the invention or of a composition according to the invention, to a subject in need thereof.

Another object of the invention is a method for preventing and/or treating imbalances and/or disorders related to the imbalance of the microbiota of the skin and/or mucous membranes (gums, periodontium, genital mucous membranes) and/or skin appendages, and/or their adnexa, whether immature, normal or mature/aged, comprising the administration, in particular the topical administration, of an effective amount of an extract according to the invention or of a composition according to the invention, to a subject in need thereof.

In particular, the composition or extract according to the invention is intended for preventing and/or treating inflammatory or irritative reactions or pathologies or disorders of the barrier or homeostasis of the skin, skin appendages (hair and nails) and/or mucous membranes (gums, periodontium, genital mucous membranes), whether immature, normal or mature/aged.

Advantageously, the inflammatory or irritative reactions, disorders or pathologies or disorders of the skin barrier or homeostasis are: acne, rosacea or erythrocouperose, vascular disorders, especially redness and couperose, diaper dermatitis, atopic dermatitis, eczema, contact dermatitis, irritant dermatitis, allergic dermatitis, seborrheic dermatitis (cradle cap), sensitive skin, reactive skin, dry skin (xerosis), dehydrated skin, skin with redness, cutaneous erythema, aged or photo-aged skin, photosensitized skin, pigmented skin, (melasma, post-inflammatory pigmentation . . . ), skin with stretch marks, sunburn, irritation caused by chemical, physical (e.g. stress for pregnant women), bacteriological or fungal agents, skin aging, particularly photoaging, and disorders related to radical attacks caused by chemical or atmospheric pollution, and/or UV or IR exposure.

Advantageously, inflammatory or irritative reactions, disorders or pathologies or disorders of the mucosal barrier or homeostasis are gingivitis (sensitive gums of newborns, hygiene problems, due to smoking or others), and irritations of the external or internal male or female genital spheres.

Advantageously, the inflammatory, irritative reactions, disorders or pathologies or disorders of the barrier or homeostasis of the skin appendages are brittle nails, fragile nails, weakened hair, brittle hair, dry hair, alopecia, seborrheic dermatitis and folliculitis dermatitis.

In a particularly advantageous way, the composition or extract according to the invention is intended as an anti-aging agent, against cutaneous aging, in particular chronological and actinic aging, for example related to sun exposure or inflammation phenomena, or to act on irritated skin.

In a preferred embodiment, the extract according to the invention is used to prevent and/or treat:

disorders or pathologies of the skin and/or mucous membranes and/or skin appendages, advantageously inflammatory reactions, disorders related to intrinsic or extrinsic stress, such as psychological stress, stress related to a state of anxiety, stress related to radical attacks related or not to chemical or atmospheric pollution, and/or related to UV or IR exposure, advantageously selected from sensitive skin, reactive skin and/or reactive mucous membranes, irritated skin, inflamed skin, barrier or homeostasis disorders, photosensitized skin, mechanical and/or thermal aggressions; and/or
vascular disorders, preferably redness and couperose; and/or
imbalances in the microbiota, advantageously selected from atopic dermatitis, eczema, development of unpleasant axillary odors, weakening of the cutaneous barrier, acne, psoriasis, hidradenitis suppurativa, folliculitis, cradle cap, dandruff, itching, particularly of the scalp, irritation, candidiasis and bacterial vaginosis, and/or for use as a healing agent.

Another object of the invention is the cosmetic use of an extract according to the invention or of a composition according to the invention in the treatment and/or prevention of dehydrated skin; skin with redness; aged skin; skin aging.

Another object of the invention is the cosmetic use of an extract according to the invention or of a composition according to the invention for the care of the skin appendages, in particular for the prevention of brittle nails; fragile nails; weakened hair; brittle hair; and dry hair.

The invention also relates to a method of cosmetic care of the skin and/or skin appendages and/or mucous membranes, in view of improving their condition and/or appearance, consisting in administering a composition or extract according to the present invention; in particular improving the firmness, elasticity or tonicity of the skin, reinforcing the mechanical properties of the skin and mucous membranes, in particular combatting withered, limp, distended and/or sagging skin, and/or reinforcing and/or restoring the elasticity or firmness of the skin, or treating fine lines and wrinkles.

In particular, the invention concerns a method for the cosmetic care of the skin and/or mucous membranes and/or skin appendages, in view of preventing damage to the skin barrier and dehydration thereof, or in view of moisturizing the skin and/or mucous membranes, consisting in applying to the skin and/or mucous membranes and/or skin appendages a composition or extract according to the present invention.

The invention also concerns a method of cosmetic care of the skin and/or skin appendages, in view of preventing aging, consisting in applying to the skin and/or skin appendages a composition or extract according to the present invention.

The invention also concerns the cosmetic use of a composition or an extract or a composition according to the invention to moisturize the skin and/or mucous membranes, to act on the elasticity or firmness of the skin, in particular as a tensor or anti-wrinkle agent, to act on sensitive skin.

In particular, the invention concerns the cosmetic use of a composition or extract according to the invention in the treatment and/or prevention of dehydrated skin; skin with redness; aged skin; evenness of complexion; reduction of spots on the skin or skin aging, in particular chronological and/or actinic aging.

Cosmetic uses and cosmetic care methods according to the present invention are typically performed on healthy skins or healthy parts of the body and are not therapeutic.

### DESCRIPTION OF FIGURES

FIG. 1 describes the measurement of NO production by keratinocytes under cortisol stress.

FIG. 2 describes the effects of holy basil extract on cellular bioenergetics in fibroblasts subjected to pollutant stress, in particular the effects on oxygen consumption FIG. 2A, ATP production FIG. 2B, lactate production FIG. 2C and NAD/NADH ratio FIG. 2D (*p<0.05; **p<0.01 vs untreated control; #p<0.05; ##p<0.01 vs pollutant).

FIG. 3 describes the percentage of damaged mitochondria per cell (%) (###p<0.0001 vs pollutant).

FIG. 4 illustrates lysosomal density (intensity/μm²) (***p<0.0001 vs control; #p<0.05; ###p<0.0001 vs pollutant).

FIG. 5: Mitophagy (% of lysosomes co-localized with damaged mitochondria) (***p<0.0001 vs control; ###p<0.0001 vs pollutant).

FIG. 6 illustrates the percentage of fibroblast migration between T0 and T40h.

### EXAMPLE 1

### Extract According to the Invention

An extract of holy basil is obtained by the following method:

a) dissolving 10% (w/w) of the aerial parts of Ocimum sanctum—purple variety—ground in a 40/60 w/w water/propanediol mixture

b) 2-hour extraction at 60° C.

c) solid/liquid separation by successive filtrations.

The polyphenol-rich liquid extract thus obtained has the following characteristics (% by weight of molecules/dry extract of the extract obtained):
Dry extract (2 hours, 105° C., ventilated oven): 1.0%
Total polyphenols: 11.4%
Flavonoids: 3.6%
Hydroxycinnamic derivatives: 7.6%

### EXAMPLE 2

### Extract According to the Invention

An extract of holy basil is obtained by the following method:

a) dissolving 10% (w/w) of the ground aerial parts of Ocimum sanctum—green variety—in a 40/60 w/w water/propanediol mixture

b) 3-hour extraction at 60° C.

c) solid/liquid separation by successive filtrations.

The polyphenol-rich liquid extract thus obtained has the following characteristics (% by weight of molecules/dry extract of the extract obtained):
Dry extract (2 h, 105° C., ventilated oven): 1.4%
Total polyphenols (determined by the Folin Ciocalteu method): 16%
Flavonoids: 3.5%
Hydroxycinnamic derivatives: 20%.

### EXAMPLE 3

### Biological Activity Tests on the Extract According to the Invention

### A. Antioxidant Activity

### A.1. Antioxidant Effect

### A.1.1 Activity on H₂O₂-Induced ROS Production in Normal Human Keratinocytes

The antioxidant activity of holy basil extract was investigated by assessing the amount of ROS (Reactive Oxygen Species) produced by keratinocytes subjected to H₂O₂-induced oxidative stress.

### a. Material and Method

Normal human keratinocytes were treated for 24 hours with six concentrations of the holy basil extract obtained according to Example 1 (0.00032% to 0.01% dm) or the antioxidant reference molecules: Quercetin at 10 μM or Vitamin C at 500 μM, before incorporation of the H2DCF-DA probe.

Keratinocytes were then stimulated with 100 μM hydrogen peroxide (H₂O₂) for 20 minutes. ROS production was assessed by measuring the fluorescence emitted by the probe on contact with ROS.

Results were statistically analyzed using a one-way ANOVA followed by a Tukey test.

### b. Results

The results are summarized in Table 2. Holy basil extract significantly inhibited, in a dose-dependent manner, the production of ROS by keratinocytes in response to H₂O₂-induced oxidative stress, thus exhibiting antioxidant activity.

TABLE 2
ROS production by keratinocytes under H₂O₂ stress

| | ROS | | |
|---|---|---|---|
| | (Fluorescence units) | Inhibition | |
| Control cells | 8286.3 ± 789.7 | | |
| Stimulated cells (H2O2) | 71122.8 ± 3400.8 | | |
| Reference (Quercetin 10 μM) | 17453.8 ± 2880.6 | 85% | p < 0.001 |
| Reference (Vitamin C 500 μM) | 7165.2 ± 586.2 | 102% | p < 0.001 |
| BS 0.00032% dm | 57775.2 ± 6693.0 | 21% | p < 0.001 |
| BS 0.00063% dm | 50526.0 ± 3546.1 | 33% | p < 0.001 |
| BS 0.00125% dm | 52697.8 ± 5770.0 | 29% | p < 0.001 |
| BS 0.0025% dm | 35211.5 ± 4842.6 | 57% | p < 0.001 |
| BS 0.005% dm | 20997.5 ± 4198.4 | 80% | p < 0.001 |
| BS 0.01% dm | 11943.2 ± 2435.9 | 94% | p < 0.001 |

### A.1.2. Activity on UVA-Induced ROS Production in Reconstructed Human Epidermis

The protective effect against oxidative stress induced by UVA irradiation was studied by ROS assay on reconstructed human epidermis.

### a. Material et Method

Reconstructed human epidermis were pre-treated with a formulation containing 3% basil extract obtained according to Example 1 or its placebo for 24 hours. The epidermis were then irradiated with 30 J/cm² of UVA.

ROS production was assessed by measuring the fluorescence emitted by the H2DCF-DA probe in contact with ROS.

### b. Results

The results are summarized in Table 3. Holy basil extract in a 3% formulation inhibited UVA-induced ROS production, demonstrating a protective effect against oxidative stress induced by UVA irradiation.

TABLE 3
ROS production in UVA-irradiated reconstructed epidermis

| | ROS | |
|---|---|---|
| | (% vs untreated control) | Inhibition |
| Epidermal controls | 100 ± 20 | |
| Placebo + UVA 30 J/cm² | 439 ± 46 | |
| Holy basilic 30% + UVA 30 J/cm² | 333 ± 77 | 31% |

### 1.3. Activity on Menadione-Induced ROS Production in Normal Human Keratinocytes and Fibroblasts

The antioxidant activity of holy basil extract was investigated by assessing the amount of ROS produced by keratinocytes or fibroblasts subjected to menadione-induced oxidative stress.

### a. Material et Method

Normal human keratinocytes, on the one hand, and normal human fibroblasts, on the other, were treated for 24 hours with the 0.001% and 0.005% dry matter (dm) holy basil extract obtained according to Example 1 or with 10 mM N-Acetyl Cysteine (NAC), a ROS scavenger molecule used as an antioxidant reference. Oxidative stress was then induced by 1 h incubation in the presence of 50 or 20 μM menadione (2-methyl-1,4-naphthoquinone).

ROS were labeled with the CellRox® probe; intracellular fluorescence, proportional to the amount of ROS, was measured.

Results were statistically analyzed using a one-factor ANOVA, followed by a Dunnett's test.

### b. Results

The results are shown in Tables 4 and 5. Holy basil extract significantly inhibited menadione stress-induced ROS production in keratinocytes (Tab.4) and fibroblasts (Tab.5), confirming its antioxidant activity.

TABLE 4
ROS production by keratinocytes subjected to Menadione stress

| | ROS | |
|---|---|---|
| | (Arbitrary units) | Inhibition |
| Control cells | 2480 | |
| Stimulated cells (Menadione 50 μM) | 4820 ± 965.1 | |
| Reference (NAC 10 mM) | 2490 ± 94.3 | 100% |
| BS 0.001% dm | 3700 ± 61.4 | 48% |
| BS 0.005% dm | 3270 ± 164.2 | 66% |

TABLE 5
ROS production by fibroblasts subjected to Menadione stress

| | ROS | | |
|---|---|---|---|
| | (Arbitrary units) | Inhibition | |
| Control cells | 906 ± 14.0 | | |
| Stimulated cells | 5283 ± 176.7 | | |
| (Menadione 200 μM) | | | |
| Reference (NAC 10 mM) | 3114 ± 14.2 | 50% | p < 0.001 |
| BS 0.001% dm | 4718 ± 162.6 | 13% | p < 0.05 |
| BS 0.005% dm | 3659 ± 273.8 | 37% | p < 0.001 |

### A.2. Lipid Peroxidation Inhibition

ROS are the main activators of lipid peroxidation, primarily targeting polyunsaturated lipids bound to the cell membrane. The anti-lipid peroxidation activity of holy basil extract was investigated in keratinocytes or fibroblasts subjected to oxidative stress induced by cumene hydroperoxide.

### a. Material and Method

Normal human keratinocytes, on the one hand, and normal human fibroblasts, on the other, were treated for 24 hours with the 0.001% and 0.005% dm holy basil extract obtained according to example 1 or with the antioxidant reference, vitamin E at 200 μM. Oxidative stress was then induced by incubation for 1 hour in the presence of cumene hydroperoxide at 200 or 50 μM.

Lipid peroxides were revealed by labelling with BODIPY® 581/591 C11; intracellular fluorescence was measured.

Results were statistically analyzed using a one-way ANOVA followed by a Tukey test.

### b. Results

The results are summarized in Tables 6 and 7. Holy basil extract significantly inhibited oxidative stress-induced lipid peroxidation in keratinocytes (tab.6) and fibroblasts (tab.7), confirming its antioxidant activity.

TABLE 6
Quantification of lipid peroxidation in keratinocytes
subjected to cumene hydroperoxide stress

| | Peroxidized lipids | | |
|---|---|---|---|
| | (Arbitrary units) | Inhibition | |
| Control cells | 4067.9 ± 8.8 | | |
| Stimulated cells | 10018.3 ± 15.7 | | |
| (Cumene 200 μM) | | | |
| Reference | 4485.3 ± 43.3 | 93% | p < 0.001 |
| (Vitamin E 200 μM) | | | |
| BS 0.001% dm | 7166.1 ± 167.8 | 46% | p < 0.001 |
| BS 0.005% dm | 6373.5 ± 253.1 | 61% | p < 0.001 |

TABLE 7
Quantification of lipid peroxidation in fibroblasts
subjected to cumene hydroperoxide stress

| | Peroxidized lipids | | |
|---|---|---|---|
| | (Arbitrary units) | Inhibition | |
| Control cells | 5310.7 ± 31.8 | | |
| Stimulated cells | 26573.8 ± 3016.4 | | |
| (Cumene 50 μM) | | | |
| Reference | 11413.4 ± 69.4 | 71% | p < 0.001 |
| (Vitamin E 200 μM) | | | |
| BS 0.001% dm | 20751.3 ± 271.5 | 27% | p < 0.05 |
| BS 0.005% dm | 18998.15 ± 625.4 | 36% | p < 0.05 |

### A.3. Effect on Antioxidant Defenses

Glutathione (GSH) is the main endogenous antioxidant peptide capable of neutralizing ROS. Intracellular GSH depletion is responsible for oxidative stress-induced damage associated with mitochondrial toxicity.

The effect of holy basil extract on intracellular glutathione stores was evaluated in fibroblasts subjected to menadione-induced oxidative stress.

### a. Material and Method

Normal human fibroblasts were treated for 24 hours with 0.001% and 0.005% dm holy basil extract obtained according to Example 1 or with the antioxidant reference, 10 mM NAC. Oxidative stress was then induced by 4-hour incubation in the presence of 25 μM menadione.

GSH stock was monitored using the fluorescent probe Monochlorobimane (Mbi); intracellular fluorescence, proportional to the amount of GSH, was measured.

### b. Results

The results are summarized in Table 8. Holy basil extract restored intracellular glutathione depleted under menadione-induced oxidative stress more significantly than the antioxidant reference (10 mM NAC), confirming the antioxidant activity of the extract.

TABLE 8
Evolution of glutathione (GSH) content in
fibroblasts subjected to Menadione stress

| | GSH | |
|---|---|---|
| | (Arbitrary units) | Increase |
| Control cells | 7299.0 ± 1549.4 | |
| Stimulated cells | 3191.4 ± 813.2 | |
| (Menadione 25 μM) | | |
| Reference | 4997.7 ± 947.2 | 44% |
| (NAC 10 mM) | | |
| BS 0.001% dm | 6263.2 ± 898.4 | 80% |
| BS 0.005% dm | 7411.34 ± 1745.3 | 103% |

### A.4. Conclusion

Taken together, these results demonstrate the antioxidant activity of holy basil extract.

### B. Anti-Inflammatory Activity

The anti-inflammatory activity of holy basil extract was investigated by assaying interleukin 8 (IL8) produced by keratinocytes in response to PMA-induced pro-inflammatory stress.

### a. Material and method

Normal human keratinocytes were pre-incubated for 24 hours in the presence of holy basil extract obtained according to Example 1 at 0.001%, 0.005% or 0.01% dm or dexamethasone at 0.1 μM, an anti-inflammatory reference. The cells were then stimulated by 24-hour treatment in the presence of 10 μg/mL PMA (Phorbol-Myristate-Acetate).

At the end of treatment, the IL8 produced was quantified by ELISA assay in the culture supernatants.

Results were analyzed statistically using the Student t-test.

### b. Results

The results are summarized in Table 9. Holy basil extract significantly inhibited PMA-induced IL8 production in keratinocytes, in some cases more significantly than the reference dexamethasone; it therefore exhibits anti-inflammatory activity.

TABLE 9
Assaying of interleukin 8 produced by keratinocytes
subjected to PMA-induced inflammatory stress

| | IL8 | | |
|---|---|---|---|
| | (pg/ml) | Inhibition | |
| Control cells | 208.7 ± 49.5 | | |
| Stimulated cells | 6806.5 ± 334.5 | | |
| (PMA 10 μg/ml) | | | |
| Reference | 4194.4 ± 814.1 | 40% | p < 0.01 |
| (dexamethasone 0.1 μM) | | | |
| BS 0.001% dm | 4399.3 ± 1936.7 | 36% | ns |
| BS 0.005% dm | 3496.4 ± 1773.0 | 50% | p < 0.05 |
| BS 0.01% dm | 4121.2 ± 1572.9 | 41% | p < 0.05 |

### C. Anti-Aging Activity

Skin aging is characterized by an alteration in the dermal matrix, with a reduction in the quality and quantity of matrix fibers, mainly collagen and elastin.

The anti-aging activity of holy basil was assessed by its ability to stimulate the expression and production of dermal matrix markers under standard conditions and under conditions of inflammatory stress modeling chronic inflammation, also known as “inflamm'aging” or low-grade inflammation.

### C.1. Effect on the Dermal Matrix

The effect of holy basil extract on the expression of dermal matrix markers was evaluated on normal human dermal fibroblasts in culture or on reconstructed human skin.

### C.1.1 Stimulation of Gene Expression for Dermal Matrix Markers in Normal Human Fibroblasts

### a. Material and Method

Normal human dermal fibroblasts were incubated for 48 hours in the presence of holy basil extract obtained according to Example 1 at 0.001% and 0.005% dm or TGFβ1 at 5 ng/ml, the positive assay reference.

Gene expression of Collagen I, Elastin, Dermatopontin and MMP1 (Matrix-Metalloproteinase-1) was assessed by real-time quantitative PCR.

Results were statistically analyzed using a one-factor analysis of variance (ANOVA), followed by a Dunnett's post-test.

### b. Results

The results are summarized in Table 10. Holy basil extract significantly stimulated gene expression of collagen I, elastin and dermatopontin, key markers involved in dermal matrix density. In addition, the extract significantly inhibited gene expression of MMP1, an enzyme involved in dermal matrix degradation.

These results demonstrate a densification of the dermal matrix and thus an anti-aging activity.

TABLE 10
Gene expression of dermal matrix markers in fibroblasts

| | | Reference | BS | BS |
|---|---|---|---|---|
| | Control | (TGFB1 | 0.001% | 0.005% |
| | cells | 5 ng/ml) | dm | dm |
| Collagen I | 1.23 ± 0.28 | 10.86 ± 4.17 | 4.58 ± 0.17 | 5.88 ± 1.41 |
| (Relative quantity | | +786% | +274% | +380% |
| and % of | | p < 0.001 | p < 0.001 | p < 0.001 |
| induction) | | | | |
| Elastin | 0.82 ± 0.27 | 67.5 ± 20.55 | 5.44 ± 3.7 | 3.59 ± 0.37 |
| (Relative quantity | | +8148% | +565% | +338% |
| and % of | | p < 0.001 | p < 0.001 | p < 0.001 |
| induction) | | | | |
| Dermatopontin | 0.85 ± 0.16 | 43.17 ± 5.97 | 2.20 ± 0.56 | 1.58 ± 0.14 |
| (Relative quantity | | +5001% | +160% | +87% |
| and % of | | p < 0.001 | p < 0.001 | p < 0.01 |
| induction) | | | | |
| MMP1 | 0.94 ± 0.37 | 0.33 ± 0.13 | 0.32 ± 0.11 | 0.21 ± 0.02 |
| (Relative quantity | | −65% | −66% | −67% |
| and % of | | p < 0.01 | p < 0.01 | p < 0.01 |
| induction) | | | | |

### C.1.2 Stimulation of Protein Expression for Dermal Matrix Markers in Normal Human Fibroblasts

### a. Material and Method

Human dermal fibroblasts were treated for 48 hours with the holy basil extract obtained according to Example 1 at 0.001% and 0.005% dm or with a mixture of Vitamin C at 10 μM and TGFβ at 5 ng/ml, used as a positive reference.

Collagen I production was assessed by immunofluorescence labeling and image analysis.

### b. Results

The results are summarized in Table 11. Holy basil extract significantly stimulated collagen I labelling in fibroblasts. This result confirms the extract's activity on dermal matrix densification.

TABLE 11
Collagen I production by fibroblasts

| | Collagen I | |
|---|---|---|
| | (Arbitrary units) | Increase |
| Control cells | 1831.5 ± 189.9 | |
| Reference | 2204.6 ± 491.0 | 20% |
| (Vit C 10 μM + | | |
| TGFB 5 ng/ml) | | |
| BS 0.001% dm | 2061.9 ± 46.3 | 13% |
| BS 0.005% dm | 2182.5 ± 79.1 | 19% |

### C.1.3 Stimulation of Protein Expression of Dermal Matrix Markers in Normal Human Fibroblasts

### a. Material and Method

A formulation (cream) containing 3% holy basil extract obtained according to Example 1, or its placebo, were applied to the surface of reconstructed human skin. After 24 hours incubation, gene expression of markers of interest for their dermal benefits was assessed by real-time quantitative PCR.

### b. Results

The results are summarized in Table 12. Holy basil extract significantly increased CLN3 and PXN gene expression in reconstructed skin.

CLN3 encodes a lysosomal/endosomal transmembrane protein (ceroid-lipofuscinosis neuronal 3) involved in regulating the autophagy process. Autophagy is an intracellular detoxification process enabling the degradation and recycling of damaged proteins and organelles. This process is essential for maintaining cell function and homeostasis. Stimulation of autophagy is of interest for slowing down aging and extending cell lifespan.

PXN encodes a focal adhesion molecule (paxillin), whose expression decreases with age and therefore represents a target for anti-aging action. Because of its crucial role in cell-matrix connections and mechanotransduction between the fibroblast and its matrix, the loss of paxillin with age could compromise cellular function, resulting in the onset or aggravation of skin aging. Inducing its expression could help restore age-related loss of collagen and dermal contractility.

TABLE 12
Genes significantly modulated by the 3% holy basil extract
formulation compared with placebo (set at 1.00)

| | | Relative | |
|---|---|---|---|
| Genes | Names | quantity | P value |
| CLN3 | Battenin/Ceroid- | 1.26 (+26%) | 0.0034 |
| | lipofuscinosis, | | |
| | neuronal 3 | | |
| PXN | Paxillin | 1.91 (+91%) | 0.0447 |

### C.2. Effect in an Inflamm'aging Model

With age, low-grade, silent but chronic inflammation can develop in skin tissue. This is the result of cumulative exposure to external stresses throughout life. This inflammation results in the release of inflammatory cytokines (IL1, IL6, TNF, CRP . . . ), capable of generating tissue damage and the production of reactive oxygen species, thus accelerating the skin's aging process. This phenomenon of cutaneous aging linked to an inflammatory process is known as Inflamm'aging. Holy basil extract was evaluated in an in vitro model reproducing the inflammaging process: inflammation, represented by stimulation of IL1α production, is induced on reconstructed epidermis. Conditioned media from these reconstructed epidermises are then applied to fibroblasts and the expression of dermal markers, altered by IL1α produced by the epidermis, is assessed.

### a. Material and Method

Reconstructed epidermises were treated topically with a formulation (cream) containing 3% holy basil extract obtained according to example 1 or its placebo, or systemically with dexamethasone at 1 μM, an anti-inflammatory reference. After 24 hours pre-incubation, the epidermis was stimulated with 0.5 μg/mL PMA and incubated again for 24 hours.

Culture supernatants were collected, an IL1α assay was performed on the one hand and, on the other, these supernatants (conditioned media) were plated onto cultures of normal human dermal fibroblasts.

After 24 hours incubation of fibroblasts in the presence of conditioned media, expression of markers of interest was assessed by real-time quantitative PCR.

Results were statistically analyzed using an ANOVA followed by a Tukey test.

### b. Results

The results are shown in Tables 13 and 14. Holy basil extract, formulated at 3%, significantly inhibited PMA-induced IL1α release on reconstructed epidermis (tab.13). This effect was significantly higher than that of placebo.

The application of conditioned media to fibroblast cultures from PMA-stimulated reconstructed epidermis induced variations in gene expression of inflammatory markers, oxidative stress, and dermal matrix synthesis and degradation (Tab. 14). These results validate the Inflamm'aging model.

Under these conditions, holy basil extract modulated the overexpression of cytokines and chemokines (CXCL1, IL8, IL6, CCL2), oxidative stress markers (MT1G, SOD2), matrix degradation enzymes (MMP1, MMP3) and ICAM1. The extract also restored expression of matrix assembly markers (HAS2, DPT).

These results demonstrate the ability of holy basil extract to modulate the inflamm'aging process.

TABLE 13
Assay of IL1α produced by PMA-stimulated
reconstructed epidermis

| | Interleukine-1 | | | |
|---|---|---|---|---|
| | alpha (pg/ml) | Inhibition | | |
| Control cells | 122 ± 4 | | | |
| Stimulated cells | 533 ± 35 | | | |
| (PMA 0.5 μg/ml) | | | | |
| Reference | 334 ± 41 | 48% | p < 0.001 | |
| Dexamethasone | | | | |
| 10 μM) | | | | |
| Placebo | 246 ± 15 | 70% | p < 0.001 | p < 0.05 |
| BS 3% | 147 ± 17 | 94% | p < 0.001 | |

TABLE 14
Genes modulated by the 3% holy basil formula in the Inflamm'aging model:
gene expression in fibroblasts incubated with conditioned media
(Level of expression in relative quantity; % change versus PMA)

| | Control | | PMA | Placebo | BS 3% |
|---|---|---|---|---|---|
| Cytokines/Chemokines | CXCL1 | 100 | 401 | 230 (−57%) | 144 (−85%) |
| | IL8 | 100 | 1645 | 325 (−85%) | 114 (−99%) |
| | IL6 | 100 | 1817 | 509 (−76%) | 264 (−90%) |
| | CCL2 | 100 | 307 | 198 (−53%) | 123 (−89%) |
| Response to oxidative | MT1G | 100 | 387 | 403 (+6%) | 182 (−71%) |
| and cellular stress | SOD2 | 100 | 333 | 241 (−39%) | 147 (−80%) |
| Extracellular matrix | HAS2 | 100 | 78 | 100 (+100%) | 121 (+196%) |
| assembly/synthesis | DPT | 100 | 62 | 94 (+83%) | 100 (+100%) |
| Degradation of the | MMP1 | 100 | 1060 | 306 (−76%) | 169 (−93%) |
| intracellular matrix | MMP3 | 100 | 533 | 386 (−34%) | 250 (−65%) |
| Cell-cell and cell-matrix | ICAM1 | 100 | 225 | 172 (−41%) | 111 (−91%) |
| interactions | | | | | |

### D. Defensive Activity Against Various Stresses

The skin is exposed daily to various types of exogenous and/or endogenous stress, which can alter its function and quality, and ultimately lead to irreversible damage.

The protective effect of holy basil extract was evaluated in various models representing different external stresses (pollution) or internal stresses (psychological stress).

### D.1. Protection Against Skin Damage Caused by Psychological Stress

Nitric oxide (NO) is a reactive species involved in the regulation of skin homeostasis. This highly unstable molecule is synthesized and released by numerous cell types to regulate physiological and homeostatic reactions, such as melanogenesis, wound healing, inflammation regulation, immunomodulation, vasodilation . . .

While low levels of NO are important for cell signaling, the release of high levels of NO is generally associated with oxidative stress.

Psychological stress is characterized by activation of the hypothalamicuitary-adrenal (HPA) axis, linking the central nervous and endocrine systems, with an increase in endogenous glucocorticoids such as cortisol.

Numerous studies have described the negative effects of psychological stress on skin homeostasis, leading to altered immune and inflammatory responses. The inflammatory response includes the activation of free radicals, including NO.

Holy basil extract was evaluated in a model reproducing the effect of psychological stress (emotional disorder/anxiety) on the skin by treating keratinocytes with cortisol and quantifying NO as a biomarker of induced stress.

### a. Material and Method

NO production by normal human epidermal keratinocytes was measured by continuous amperometry, for 10 minutes, after treatment with 1 μM cortisol in the presence of the holy basil extract obtained according to example 1 at 0.001% and 0.005% dm or 1 mM ascorbic acid (AA) (positive reference).

### b. Results

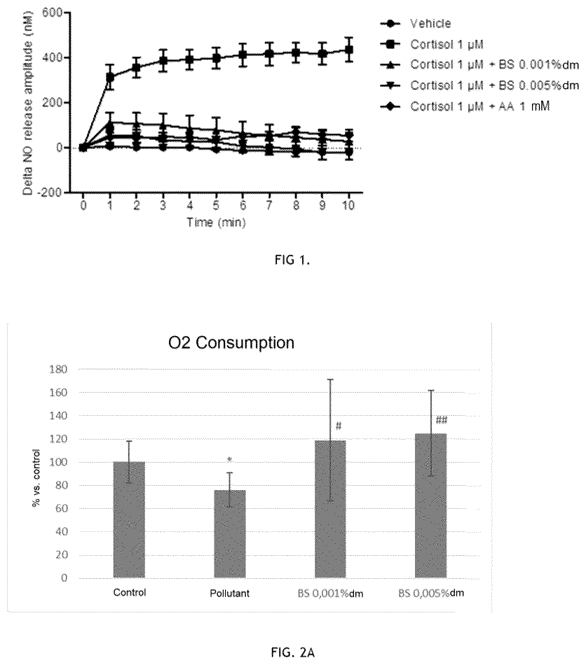

The results are shown in FIG. 1. Holy basil extract significantly inhibited NO production induced in keratinocytes by cortisol stress. The extract therefore counterbalances the adverse effects of emotional/psychological stress on skin homeostasis.

### D.2. Protection Against DNA Damage

Atmospheric pollutants have negative effects on many tissues, including the skin. Repeated exposure to pollutants can contribute to premature aging, depigmentation and DNA damage. The protective effect of holy basil extract was evaluated on DNA damage (double-strand breaks) induced in keratinocytes by pollutant stress.

### a. Material and Method

Normal human epidermal keratinocytes were pre-incubated for 24 hours in the presence of the holy basil extract obtained in Example 1 at 0.001% and 0.005% dm or the reference nicotinamide, prior to 1-hour exposure to camptothecin. Camptothecin is a genotoxic compound, a toposiomerase I inhibitor, used for the induction of DNA double-strand breaks.

Double-strand breaks were detected by immunostaining of γH2AX (phosphorylation of histone H2AX which intervenes early in response to double-strand breaks) and quantified by image analysis.

Results were statistically analyzed using an ANOVA followed by a Tukey test.

### b. Results

The results are shown in Table 15. Holy basil extract significantly inhibited double-strand DNA breaks induced by camptothecin treatment. The extract therefore exhibits protective activity against genotoxic pollutant stress.

TABLE 15
Expression of γH2AX in keratinocytes under camptothecin stress

| | Cy5 | | |
|---|---|---|---|
| | (Intensity) | Inhibition | |
| Control cells | 3158.4 ± 33.2 | | |
| Stimulated cells | 4791.9 ± 111.7 | | |
| (Camptothecin) | | | |
| Reference | 3052.8 | −106% | |
| (Nicotinamide) | | | |
| BS 0.001% dm | 3870.5 ± 207.9 | −56% p | < 0.01 |
| BS 0.005% dm | 3328.4 ± 83.5 | −90% p | < 0.05 |

### D.3. Protection of Mitochondrial Function Against Pollutant Stress

The protective effect of holy basil extract against urban pollution was investigated in normal human fibroblasts. Cells were exposed to the urban pollutant in the presence of the extract, and cellular bioenergetics were analyzed using BBS+ technology, as well as the occurrence of autophagy/mitophagy.

Alteration of mitochondrial function plays a central role in the aging process; 90% of age-related ROS production is mitochondrial in origin.

Environmental factors (UV, pollution) can slow cell function and increase oxidative stress, contributing to premature aging.

Intracellular ROS are mainly formed by dysfunction of the mitochondrial electron transfer chain. Mitochondrial DNA is particularly sensitive to ROS, the following phenomena which ultimately contribute to the aging process:
reduced energy production,
increased oxidative stress,
increased mitochondrial function.

### D.3.1 Studying Cellular Bioenergetics

### a. Material and method

Normal human dermal fibroblasts were incubated for 120 minutes in the presence of the 0.1% urban pollutant mixture and the 0.001% and 0.005% dm holy basil extract obtained in Example 1. Cellular bioenergetics were assessed using the BBS+ (Bioenergetic Balance Screen Plus) method, which involves multiplexed quantification of oxygen consumption, ATP production, extracellular lactate levels and the NAD+/NADH ratio.

Results were analyzed statistically using the Student t-test.

### b. Results

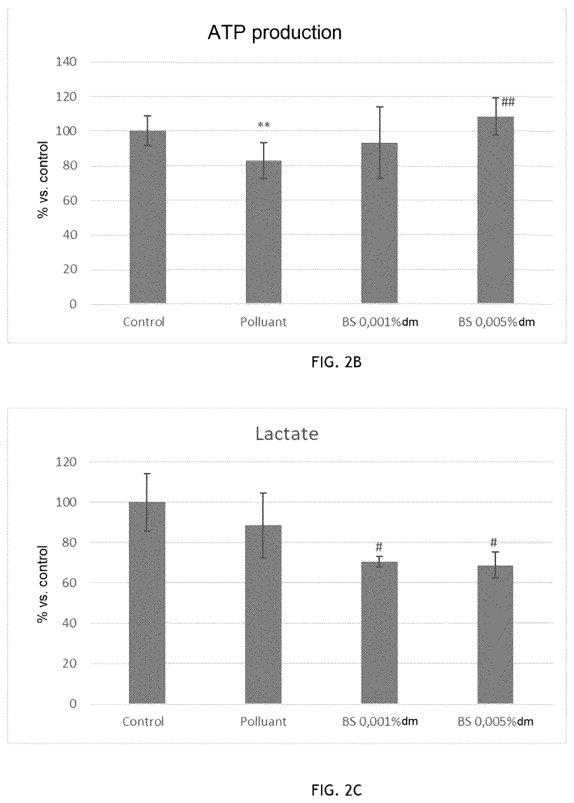

The results are shown in FIG. 2. Exposure of cells to urban pollutants induced an alteration in the respiratory chain of electron transport, resulting in a decrease in oxygen consumption and ATP levels.

Under these conditions, holy basil extract dose-dependently increased the rate of oxygen consumption, while stimulating ATP production. These observations demonstrate a direct effect of the extract on the stimulation and normalization of Krebs cycle function and energy production.

The extract is also capable of resolving pollutant-induced alterations to the mitochondrial respiratory chain. Finally, the reduction in lactate production by the extract shows that it reduces the contribution of de novo glycolysis to ATP production, while re-engaging respiratory chain activity in cellular bioenergetics.

Holy basil extract helps normalize the energy balance altered by pollutant stress.

### D.3.2 Studying Autophagy/Mitophagy

### a. Material and Method

Normal human dermal fibroblasts were incubated for 120 minutes in the presence of the 0.1% urban pollutant mixture and the 0.001% and 0.005% dm holy basil extract obtained in Example 1. Analysis of autophagy and mitophagy was assessed via a lysosomal marker and a dye specific for damaged mitochondria: Damaged mitochondria were visualized by a red fluorescence produced by reaction of a fluorochrome covalently bound to the mitochondrial membrane that emits when mitochondrial membrane damage occurs. A lysosomal dye was used to label lysosomes and assess colocalization of damaged mitochondria to lysosomes.

Three parameters were analyzed:

percentage of damaged mitochondria per cell;
the density of lysosomes per cell (lysosomal index representative of autophagy);
percentage of lysosomes including damaged litochondria (mitophagic index representative of mitophagy).

Results were analyzed statistically using the Student t-test.

### b. Results

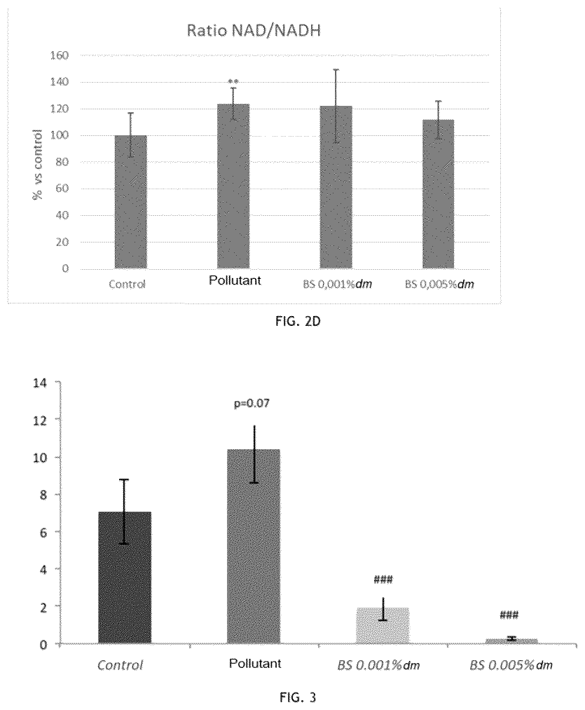

The results are shown in FIGS. 3, 4 and 5. Exposure of cells to urban pollutants increased the number of damaged mitochondria. Holy basil extract strongly and significantly inhibited the number of damaged mitochondria (FIG. 3).

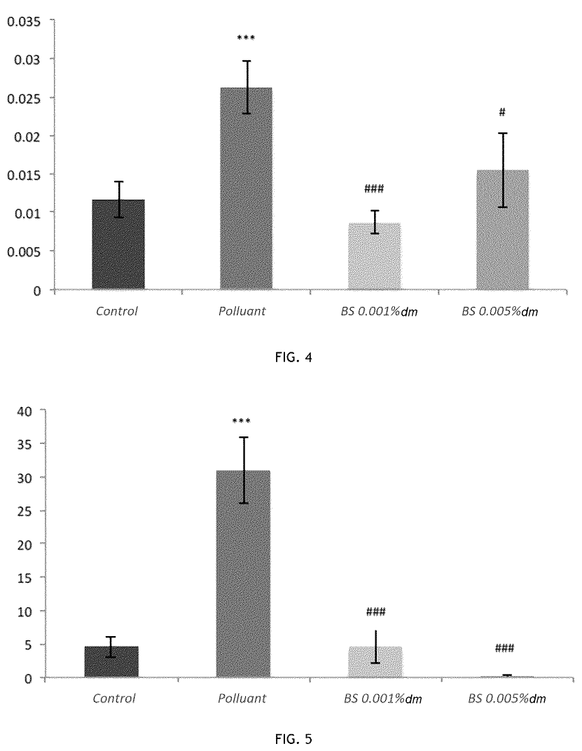

During macro-autophagy, autophagosomes are formed to transport recycled damaged mitochondrial membranes to the lysosomal compartment. Pollutant stress significantly increased the density of lysosomes per cell, showing an increase in the autophagy process. Holy basil extract reduced and normalized lysosomal density. (FIG. 4).

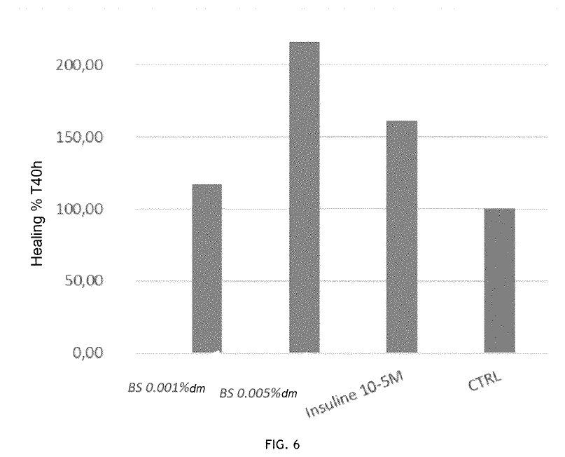

Mitophagy is represented by the percentage of lysosomes co-localized with a signal of damaged mitochondria. Pollution increased the frequency of lysosomes containing damaged mitochondria, demonstrating an increase in the mitophagy process. Pollution increases mitochondrial and endoplasmic reticulum stress, both of which induce mitochondrial membrane damage. The increase in mitophagy observed under stress is linked to cells trying to eliminate damaged mitochondria and/or malformed proteins or lipids. Holy basil extract reduced and normalized pollutant stress-induced mitophagy (FIG. 5).

Overall, holy basil extract reversed the effect of pollution on mitochondrial damage and normalized autophagy and mitophagy processes. These results demonstrate the protective activity of the mitochondrial network against external stress.

### E. Pro-Healing Activity

The pro-healing activity of holy basil extract was studied by assessing fibroblast migration in a scratch-assay model.

### a. Material and Method

A mechanical scratch was made on a confluent mat of normal human dermal fibroblasts. Immediately after the “scar” was made, the holy basil extract obtained in Example 1 at 0.001% and 0.005% dm or the positive control, insulin at 10⁻⁵ M, were added to the culture medium and cell migration monitored over time by immunofluorescent labeling of cell nuclei. Percentage migration was calculated.

### b. Results

The results are shown in FIG. 6. Holy basil extract at 0.005% stimulated fibroblast migration, indicating the extract's pro-healing activity.

### F. Moisturizing Activity

The effect of holy basil extract was evaluated on its ability to induce aquaporin-3 production in keratinocytes.

Hydration is an important criterion in preventing damage to the skin's barrier function. Aquaporin-3 plays an important role in preserving skin hydration: this transmembrane protein is involved in transporting water and glycerol into keratinocytes to maintain epidermal hydration.

### a. Material and Method

Normal human epidermal keratinocytes were incubated for 24 hours in the presence of the holy basil extract obtained in Example 1 at 0.001% and 0.005% dm or retinoic acid, positive reference. Aquaporin-3 expression was assessed by immunostaining and quantified by image analysis.

### b. Results

The results are shown in Table 16. Holy basil extract at 0.001% dm significantly increased aquaporin-3 expression in keratinocytes. This result reveals the moisturizing activity of the extract.

TABLE 16
Aquaporin-3 production in keratinocytes

| | Aquaporin-3 | |
|---|---|---|
| | (% of positive cells) | Increase |
| Control cells | 0.223 ± 0.099 | |
| Reference (Retinoic acid) | 0.323 ± 0.004 | +45% |
| BS 0.001% dm | 0.266 ± 0.053 | +19% |
| BS 0.005% dm | 0.239 ± 0.044 | +7% |

### G. Activity on Cutaneous Microbiota

The effect of holy basil extract was studied on different bacterial strains representative of the cutaneous microbiota, more specifically on their ability to form a biofilm.

### a. Material and Methods

Bacterial strains studied:

Staphylococcus epidermidis MFP04;
Staphylococcus aureus MFP03;
Corynebacterium xerosis CIP 100653T/ATCC373;
Micrococcus luteus 0116.

### a1. Studying the Extract's Effect on Bacterial Growth

The effect of the 1:50 dilution of the holy basil extract obtained in Example 1 on the growth of the 6 bacterial models was studied by monitoring growth under continuous agitation over 24 hours (S. aureus, S. epidermidis) or 48 hours (M. luteus, C. xerosis), using a multi-well incubator/plate reader. Generation times of bacterial strains were measured, using the wild-type strain as a reference. Statistical differences were determined using the Mann-Whitney test.

### a2. Studying the Effect on Biofilm Formation

Biofilm production of bacterial species was studied in multi-well plates using the crystal violet staining technique. Results were normalized and expressed as a percentage of the biofilm formation value in control media. Statistical differences were determined using the Mann-Whitney test.

### b. Results

The results are shown in Tables 17 and 18. Holy basil extract increases the generation time of a pathogenic strain (S. aureus), without significantly altering the growth of commensal strains (Tab. 17).

Furthermore, holy basil extract strongly and significantly altered the ability of S. aureus to form a biofilm (tab.18), without altering the ability of the other strains studied to form a biofilm.

These results demonstrate the extract's ability to preserve the balance of skin microbiota.

TABLE 17
Effect of holy basil extract on generation times of different bacterial strains
Generation G time (minutes)

| | S. aureus | S. epidermidis | M. luteus | |
|---|---|---|---|---|
| | MFP03 | MFP04 | 0116 | C. xerosis |
| Wild-type strain | 75.06 ± 0.23 | 8.08 ± 3.49 | 46.32 ± 15.76 | 596.28 ± 213.60 |
| Strain + BS 1/50 | 91.26 ± 2.85 | 82.73 ± 4.80 | 69.54 ± 14.22 | 722.15 ± 88.48 |
| | p < 0.05 | NS | p < 0.05 | NS |

TABLE 18
Effect of holy basil extract on biofilm formation of different bacterial strains
Biofilm (%)

| | S. aureus | S. epidermidis | M. luteus | |
|---|---|---|---|---|
| | MFP03 | MFP04 | 0116 | C. xerosis |
| Wild-type strain | 100 ± 1.89 | 100 ± 12.19 | 100 ± 29.23 | 100 ± 47.37 |
| Strain + BS 1/50 | 37.05 ± 6.23 | 104.14 ± 19.51 | 51.32 ± 13.27 | 58.08 ± 11.08 |
| | p < 0.05 | NS | NS | NS |

### Example 4 Clinical Study on the Extract According to the Invention

The clinical anti-aging efficacy of holy basil extract according to the present invention was evaluated by an in vivo clinical study. For this purpose, a formulation (cream) containing 3% holy basil extract (hereinafter referred to as active) obtained according to Example 1, or its placebo, was evaluated.

### A. Protocol

The study was a double-blind, placebo-controlled study. Subjects applied cream containing holy basil extract (active) or placebo (randomized at inclusion) for 56 days, twice a day (morning and evening).

The composition of the placebo was as follows:

TABLE 19
Placebo composition

| INCI name | % Matter |
|---|---|
| AQUA | 92.695600 |
| SCLEROTIUM GUM | 1.000000 |
| CAPRYLYL GLYCOL | 0.300000 |
| 1,2-HEXANEDIOL | 1.000000 |
| CAPRYLIC/CAPRIC TRIGLYCERIDE | 5.000000 |
| CITRIC ACID | 0.004400 |
| | 100.000000 |

The inclusion criteria defined for the subjects recruited were:
Gender: Female
Age: between 40 and 60 years
Phototype: I to III
Type: Caucasian
Subject with wrinkles (crow's feet, forehead)
No cosmetic products between day 6 and day 0.

Efficacy was assessed using the following techniques (measurements at D0, D28 and D56):
Image analysis—Topography
Measurement of biomechanical skin properties
Cross-polarized light photography.

### B. Evaluating the Anti-Aging Effect on Wrinkles

### a. Results on Crow's Feet Wrinkles

The results obtained at D0, D28 and D56 by topography analysis are listed in the table below. The active ingredient significantly reduced the area and length of the wrinkle detected at D56, with a significant difference compared with placebo.

TABLE 20
measurement results - crow’s feet wrinkles

| | | | | | | | | Active vs Placebo | | | |
|---|---|---|---|---|---|---|---|---|---|---|---|
| | | Active and Placebo results | | | | | | | | | Δ% |
| | | ΔD 28/D 0 | ΔD 56/D 0 | p-value: | | p-value: | | | | | Active vs |
| Parameter | Product | % | % | D 28 vs D 0 | | D 56 vs D 0 | | Time | p-value | | Placebo |
| Area | Active | −8.23% | −15.2% | 0.204 | ^{∘} | 0.005 | ^{∘} | D 28 | 0.378 | ^{∘∘} | −6.08% |
| (mm²) | Placebo | −2.15% | −2.04% | 0.631 | ^{∘} | 0.800 | * | D 56 | 0.029 | ** | −13.16% |
| Length | Active | −8.1% | −15.65% | 0.194 | ^{∘} | 0.003 | ^{∘} | D 28 | 0.340 | ^{∘∘} | −6.49% |
| (mm) | Placebo | −1.61% | −1.77% | 0.718 | ^{∘} | 0.823 | * | D 56 | 0.020 | ** | −13.88% |

^{∘} Matched Student t-test
^{∘∘} Unmatched Student-test
* Wilcoxon signed-rank test
** Mann-Whitney U-test

### b. Results on Forehead Wrinkles

The results obtained at D0, D28 and D56 by topography analysis are listed in the table below. On the overall area: the active ingredient significantly improved forehead wrinkles overall at D28, with no change at D56. The placebo did not induce any significant variation at D28 and induced a significant overall worsening at D56. Overall, there were significant differences in favor of the active ingredient at D28 and D56 for all topographical parameters assessed.

TABLE 21
measurement results - forehead wrinkles

| | | | | | | | | Active vs Placebo | | | |
|---|---|---|---|---|---|---|---|---|---|---|---|
| | | Active and Placebo results | | | | | | | | | Δ% |
| | | ΔD 28/D 0 | ΔD 56/D 0 | p-value: | | p-value: | | | | | Active vs |
| Parameter | Product | % | % | D 28 vs D 0 | | D 56 vs D 0 | | Time | p-value | | Placebo |
| Rxa (mm) | Active | −7.76% | −0.87% | 0.007 | ^{∘} | 0.707 | ^{∘} | D 28 | 0.120 | ^{∘∘} | −5.64% |
| | Placebo | −2.12% | 6.24% | 0.332 | ^{∘} | 0.027 | ^{∘} | D 56 | 0.043 | ^{∘∘} | −7.11% |
| Rya (mm) | Active | −7.35% | −0.89% | 0.009 | ^{∘} | 0.696 | ^{∘} | D 28 | 0.123 | ^{∘∘} | −5.36% |
| | Placebo | −1.99% | 5.45% | 0.326 | ^{∘} | 0.066 | ^{∘} | D 56 | 0.081 | ^{∘∘} | −6.34% |
| Rxq (mm) | Active | −7.65% | −0.56% | 0.011 | ^{∘} | 0.821 | ^{∘} | D 28 | 0.083 | ^{∘∘} | −6.33% |
| | Placebo | −1.32% | 7.31% | 0.523 | ^{∘} | 0.014 | ^{∘} | D 56 | 0.034 | ^{∘∘} | −7.87% |
| Ryq (mm) | Active | −7.23% | −1.06% | 0.014 | ^{∘} | 0.651 | ^{∘} | D 28 | 0.104 | ^{∘∘} | −5.74% |
| | Placebo | −1.49% | 6.5% | 0.448 | ^{∘} | 0.031 | ^{∘} | D 56 | 0.041 | ^{∘∘} | −7.56% |
| Spa (mm) | Active | −7.53% | −0.57% | 0.006 | ^{∘} | 0.802 | ^{∘} | D 28 | 0.086 | ^{∘∘} | −5.85% |
| | Placebo | −1.68% | 6.16% | 0.409 | ^{∘} | 0.028 | ^{∘} | D 56 | 0.053 | ^{∘∘} | −6.73% |
| Spq (mm) | Active | −7.55% | −0.75% | 0.013 | ^{∘} | 0.764 | ^{∘} | D 28 | 0.065 | ^{∘∘} | −6.57% |
| | Placebo | −0.98% | 7.4% | 0.613 | ^{∘} | 0.011 | ^{∘} | D 56 | 0.026 | ^{∘∘} | −8.15% |
| Sdev | Active | 0% | 0% | 0.216 | * | 0.585 | * | D 28 | 0.862 | ** | 0.00% |
| | Placebo | 0% | 0.01% | 0.367 | ^{∘} | <0.001 | * | D 56 | 0.006 | ** | −0.01% |

^{∘} Matched Student test
^{∘∘} Unmatched Student test
* Wilcoxon signed-rank test
** Mann-Whitney U-test

### d. Conclusion—Anti-Wrinkle Effect

The results show an anti-wrinkle effect, significantly different from placebo, for crow's feet wrinkles (improvement in wrinkle area and length) and forehead wrinkles (improvement in topographical parameters).

### C. Assessing the Anti-Aging Effect on the Skin's Biomechanical Properties

The results obtained at D0, D28 and D56 by Cutometry are listed in the table below.

TABLE 22
Cutometry test results

| | | | | | | | | Active vs Placebo | | | |
|---|---|---|---|---|---|---|---|---|---|---|---|
| | | Active vs Placebo | | | | | | | | | Δ% |
| | | ΔD 28/D 0 | ΔD 56/D 0 | p-value: | | p-value: | | | | | Active vs |
| Parameter | Product | % | % | D 28 vs D 0 | | D 56 vs D 0 | | Time | p-value | | Placebo |
| Ur: | Active | 6.12% | 2.48% | 0.318 | ^{∘} | 0.539 | ^{∘} | D 28 | 0.676 | ^{∘∘} | 3.17% |
| immediate | Placebo | 2.95% | −11.5% | 0.439 | ^{∘} | 0.001 | ^{∘} | D 56 | 0.006 | ^{∘∘} | 13.98% |
| tonicity | | | | | | | | | | | |
| Ua: return | Active | 6.62% | 3.98% | 0.211 | ^{∘} | 0.239 | ^{∘} | D 28 | 0.612 | ^{∘∘} | 3.16% |
| capacity | Placebo | 3.46% | −8.01% | 0.179 | ^{∘} | 0.013 | ^{∘} | D 56 | 0.008 | ^{∘∘} | 11.99% |
| Ua/Uf: | Active | 7.37% | 10.55% | 0.041 | ^{∘} | 0.001 | ^{∘} | D 28 | 0.403 | ^{∘∘} | 4.12% |
| biological | Placebo | 3.25% | −3.37% | 0.320 | ^{∘} | 0.203 | ^{∘} | D 56 | <0.001 | ^{∘∘} | 13.92% |
| elasticity | | | | | | | | | | | |
| Ur/Ue: net | Active | 4.48% | 6.14% | 0.385 | ^{∘} | 0.178 | ^{∘} | D 28 | 0.621 | ^{∘∘} | 3.42% |
| elasticity | Placebo | 1.06% | −8.85% | 0.823 | ^{∘} | 0.059 | ^{∘} | D 56 | 0.021 | ^{∘∘} | 14.99% |
| Ur/Uf: | Active | 6.6% | 7.62% | 0.187 | ^{∘} | 0.056 | ^{∘} | D 28 | 0.584 | ^{∘∘} | 3.79% |
| gross | Placebo | 2.81% | −7.58% | 0.546 | ^{∘} | 0.060 | ^{∘} | D 56 | 0.007 | ^{∘∘} | 15.20% |
| elasticity | | | | | | | | | | | |
| viscoelastic | Active | 8.69% | 10.74% | 0.056 | ^{∘} | 0.003 | ^{∘} | D 28 | 0.365 | ^{∘∘} | 5.57% |
| recovery | Placebo | 3.12% | −5.08% | 0.432 | ^{∘} | 0.113 | ^{∘} | D 56 | 0.001 | ^{∘∘} | 15.82% |
| Q2: elastic | Active | 8.51% | 9.7% | 0.184 | ^{∘} | 0.061 | ^{∘} | D 28 | 0.579 | ^{∘∘} | 4.94% |
| recovery - | Placebo | 3.57% | −9.58% | 0.545 | ^{∘} | 0.063 | ^{∘} | D 56 | 0.008 | ^{∘∘} | 19.28% |
| ↑ | | | | | | | | | | | |
| Q3: | Active | 8.9% | 11.96% | 0.043 | ^{∘} | 0.011 | ^{∘} | D 28 | 0.286 | ^{∘∘} | 6.32% |
| viscous | Placebo | 2.58% | 0.09% | 0.489 | ^{∘} | 0.978 | ^{∘} | D 56 | 0.041 | ^{∘∘} | 11.87% |
| recovery - | | | | | | | | | | | |
| ↑ | | | | | | | | | | | |

^{∘} Matched Student test
^{∘∘} Unmatched Student test
* Wilcoxon signed-rank test
** Wilcoxon signed-rank test

The active ingredient improved tonicity (Ur and Ua) at D56, whereas the placebo caused a significant worsening. The difference between active and placebo was significant in favor of active for both parameters.

The active ingredient improved Ur/Uf gross elasticity at D56. The difference is significant compared with placebo, which causes a worsening.

The active ingredient significantly improved Ua/Uf biological elasticity at D28 and D56. The difference is significant compared with placebo at D56, which causes a worsening.

The active ingredient improves net elasticity Ur/Ue at D56. The difference was significant compared with placebo at D56, which caused a worsening.

The active ingredient improved Q1 viscoelastic recovery at D28 and significantly at D56. The difference was significant compared with placebo at D56, which caused a worsening.

The active ingredient improved Q2 elastic recovery at D56. The difference was significant compared with placebo at D56, which caused a worsening.

The active ingredient significantly improved Q3 viscous recovery at D28 and D56. The difference was significant compared with placebo at D56, which did not show any significant variation.

### Conclusion—Biomechanical Properties

The active ingredient significantly improves the skin's biomechanical properties compared to placebo, with an improvement in skin tonicity and elasticity.

### D. Evaluating the Anti-Aging Effect on Complexion: Color and Evenness Measured on Photographs

The results obtained at D0, D28 and D56 by image analysis are listed in the table below.

TABLE 23
results of color and homogeneity measurements on photographs

| | | | | | | | | Active vs Placebo | | | |
|---|---|---|---|---|---|---|---|---|---|---|---|
| | | Active vs Placebo | | | | | | | | | Δ% |
| | | ΔD 28/D 0 | ΔD 56/D 0 | p-value: | | p-value: | | | | | Active vs |
| Parameter | Product | % | % | D 28 vs D 0 | | D 56 vs D 0 | | Time | p-value | | Placebo |
| a* | Active | −4.36% | −5.85% | 0.002 | ^{∘} | 0.001 | ^{∘} | D 28 | 0.051 | ^{∘∘} | −2.95% |
| | Placebo | −1.41% | −3.78% | 0.065 | ^{∘} | 0.003 | ^{∘} | D 56 | 0.315 | ^{∘∘} | −2.07% |
| Hb | Active | −0.35% | −1.31% | 0.839 | ^{∘} | 0.526 | ^{∘} | D 28 | 0.064 | ^{∘∘} | −3.84% |
| | Placebo | 3.49% | 1.26% | 0.002 | ^{∘} | 0.408 | ^{∘} | D 56 | 0.315 | ^{∘∘} | −2.57% |
| H76 | Active | −1.03% | −5.2% | 0.704 | ^{∘} | 0.044 | ^{∘} | D 28 | 0.305 | ^{∘∘} | −3.33% |
| | Placebo | 2.3% | 0.79% | 0.171 | ^{∘} | 0.692 | ^{∘} | D 56 | 0.060 | ^{∘∘} | −5.99% |

^{∘} Matched Student test
^{∘∘} Unmatched Student test
* Wilcoxon signed-rank test
** Mann-Whitney U-test

The active ingredient significantly reduced redness a* at D28 and D56. The difference is in favor of the active ingredient at D28 and D56 compared with the placebo, which also significantly reduces redness at D28 and D56.

The active ingredient reduces hemoglobin at D28 and D56.

The active ingredient significantly improves skin tone evenness from H76 to D56.

### Conclusion—Complexion Evaluation

The active ingredient significantly improves luminosity, redness and evenness of skin complexion.

### E. General Summary of Clinical Study Results

Anti-aging effect on wrinkles:

The active ingredient has an instrumentally observed anti-aging effect on wrinkles in the upper part of the face, i.e., crow's feet and forehead wrinkles. Significant differences compared to placebo were observed over time in favor of the active ingredient.
Anti-aging effect on biomechanical properties:

The active ingredient significantly improved skin tonicity and elasticity at D28 and D56, depending on the parameters considered, with significant differences from placebo in favor of the active ingredient.
Effect on complexion color and evenness:

The active ingredient improved skin tone homogeneity at D56, with a difference compared to placebo in favor of the active ingredient.

1.-15. (canceled)

16. A polyphenol-rich extract of aerial parts of holy basil, Ocimum sanctum, comprising at least 6% by weight of polyphenols relative to the total weight of the dry extract, at least 5% by weight of hydroxycinnamic derivatives relative to the total weight of the dry extract and at least 2.5% by weight of flavonoids, expressed as rutin equivalent, relative to the total weight of the dry extract.

17. The extract according to claim 16, wherein the extract comprises at least 9% by weight of polyphenols, relative to the total weight of the dry extract.

18. The extract according to claim 16, wherein the extract comprises at least 7% by weight of hydroxycinnamic derivatives relative to the total weight of the dry extract.

19. The extract according to claim 16, wherein the extract comprises at least 3.5% by weight of flavonoids, expressed as rutin equivalent, relative to the total weight of the dry extract.

20. The extract according to claim 16, wherein the extract is obtained by solid/liquid extraction of aerial parts of holy basil, in a solvent chosen from binary mixtures of water/glycol or water/glycerol.

21. A method for preparing a polyphenol-rich extract of aerial parts of holy basil, Ocimum sanctum, comprising at least 6% by weight of polyphenols relative to the total weight of the dry extract, at least 5% by weight of hydroxycinnamic derivatives relative to the total weight of the dry extract and at least 2.5% by weight of flavonoids, expressed as rutin equivalent, relative to the total weight of the dry extract, wherein the method comprises at least one solid/liquid extraction step in a solvent selected from binary mixtures of water/glycol or water/glycerol.

22. The method according to claim 6, wherein the method comprises the following successive steps:
a) drying aerial parts of holy basil and grinding the dried aerial parts of holy basil;
b) extracting the ground aerial parts in a solvent selected from binary mixtures of water/glycol or water/glycerol;
c) separating the solid phase from the liquid phase by decantation and/or centrifugation and/or successive filtrations; and
d) optionally, drying the extract obtained in step c).

23. The method according to claim 6, wherein the method further comprises a vacuum evaporation or deodorization step.

24. The method according to claim 6, wherein the glycol is propanediol or propylene glycol.

25. A composition comprising, as active ingredient, a polyphenol-rich extract of aerial parts of holy basil, Ocimum sanctum, comprising at least 6% by weight of polyphenols relative to the total weight of the dry extract, at least 5% by weight of hydroxycinnamic derivatives relative to the total weight of the dry extract and at least 2.5% by weight of flavonoids, expressed as rutin equivalent, relative to the total weight of the dry extract and a suitable excipient.

26. A method for preventing and/or treating:
disorders or pathologies of the skin and/or mucous membranes and/or skin appendages; and/or
vascular disorders; and/or
imbalances in the microbiota;
and/or for healing the skin,
comprising administering to a subject in need thereof an effective amount of a polyphenol-rich extract of aerial parts of holy basil, Ocimum sanctum, comprising at least 6% by weight of polyphenols relative to the total weight of the dry extract, at least 5% by weight of hydroxycinnamic derivatives relative to the total weight of the dry extract and at least 2.5% by weight of flavonoids, expressed as rutin equivalent, relative to the total weight of the dry extract.

27. A method for improving the condition and/or appearance of the skin and/or skin appendages and/or mucous membranes, consisting of administering a polyphenol-rich extract of aerial parts of holy basil, Ocimum sanctum, comprising at least 6% by weight of polyphenols relative to the total weight of the dry extract, at least 5% by weight of hydroxycinnamic derivatives relative to the total weight of the dry extract and at least 2.5% by weight of flavonoids, expressed as rutin equivalent, relative to the total weight of the dry extract.

28. The method according to claim 27, for moisturizing the skin and/or mucous membranes, and/or for preventing damage to the skin barrier and dehydration the skin and/or for preventing skin ageing.

29. The method according to claim 27, for improving the firmness, elasticity or tonicity of the skin, reinforcing the mechanical properties of the skin and mucous membranes.

30. The extract according to claim 20, wherein the solvent is chosen from binary mixtures of water/glycol or water/glycerol in a proportion of between 30% and 90% glycol or glycerol in water.

31. The method according to claim 21, wherein the solvent of the at least one solid/liquid extraction step is selected from binary mixtures of water/glycol or water/glycerol in a proportion of between 30% and 90% of glycol in water or glycerol in water.

32. The method according to claim 26, wherein the disorders or pathologies of the skin and/or mucous membranes and/or skin appendages are inflammatory reactions, disorders related to intrinsic or extrinsic stress, barrier or homeostasis disorders, photosensitized skin, or mechanical and/or thermal aggressions.

33. The method according to claim 26, wherein the disorders or pathologies of the skin and/or mucous membranes and/or skin appendages are sensitive skin, reactive skin and/or reactive mucous membranes, irritated skin, inflamed skin, barrier or homeostasis disorders, photosensitized skin, or mechanical and/or thermal aggressions.

34. The method according to claim 26, wherein the vascular disorders are redness and couperose.

35. The method according to claim 26, wherein the imbalances in the microbiota are selected from atopic dermatitis, eczema, development of unpleasant axillary odors, weakening of the cutaneous barrier, acne, psoriasis, hidradenitis suppurativa, folliculitis, cradle cap, dandruff, itching, irritation, candidiasis and bacterial vaginosis.

36. The method according to claim 27, for combatting withered, limp, distended and/or sagging skin, and/or reinforcing and/or restoring the elasticity or firmness of the skin, or treating fine lines and wrinkles.

37. The method according to claim 27, for treating and/or preventing dehydrated skin, skin with redness, aged skin, spots on the skin or skin aging, and/or improving evenness of complexion.
